(19) United States
(12) Patent Application Publication
(10) Pub. No.: US 2004/0219173 A1
(43) Pub. Date: Nov. 4, 2004

(54) METHOD FOR TREATING HIV

(76) Inventor: Ralph L. Bass, Chapel Hill, NC (US)

Correspondence Address: MOORE & VAN ALLEN, PLLC, 2200 W MAIN STREET, SUITE 800, DURHAM, NC, 27705, US

(21) Appl. No.: 09/721,131

(22) Filed: Nov. 22, 2000

(51) Int. Cl. A61K 9/00

(52) U.S. Cl. 424/400

(57) ABSTRACT

### Abstract of the Disclosure

A method for the treatment of humans having HIV infection. Sodium chloride in a solid formulation or sodium chloride a solution formulation is used.

### Detailed Description of the Invention

### Background of Invention

The present invention relates, in general, to a method for the treatment of HIV, namely Human Immunodeficiency Virus, the causative agent of Acquired Immune Deficiency Syndrome (AIDS). More particularly, the present invention relates to a method for treating a human person infected with HIV comprising administering to the human person a treatment effective amount of sodium chloride.

HIV, which causes AIDS, exerts a profound effect on helper/inducer T-cells, devastating the function of the immune system. Presently, a small number of drugs, for instance, zidovudine which is chemically known as 3'-azido-3'-deoxythymidine (AZT) which is a pyrimidine nucleoside analogue and also lamivudine which is chemically known as levo 2',3'-dideoxy,3'-thyacytidine (3TC) which is another pyrimidine nucleoside analogue, are either being used therapeutically or being tested in antiviral chemotherapy for suppression of the AIDS virus. The failure of various antiviral chemotherapies can be attributed partially to a lack of selective toxicity, partially to the development of chronic resistance to antiviral drugs, and partially to recurrence of infection after drug therapy has terminated.

More specifically, U.S. Patent No. 4,879,277 issued November 7, 1989 to Mitsuya, Broder, and Yarchoan, assignors to the United States of America as represented by the Department of Health and Human Services, discloses a composition of 2',3'-dideoxycytidine, a salt thereof, or an ester thereof in a pharmaceutically acceptable carrier for use in treating HIV infection. The preferred carrier is normal saline (i.e., an aqueous solution of sodium chloride). The composition may be administered intravenously, orally, nasally, rectally, or vaginally.

Also, of interest is U.S. Patent No. 5,213,803 issued May 25, 1993 to Pollock and Docherty, assignors to Northeastern Ohio Universities College of Medicine. This patent discloses a method for killing an envelope virus in vitro causing AIDS and/or Herpes infections by contacting a surface or cavity which is infected with the envelope virus with a formulation of humectant, inorganic monovalent anions, and detergent. The monovalent anions can include sodium bicarbonate, sodium thiocyanate, sodium fluoride, and sodium chloride.

Additionally of background interest, Fisher in "Evaluation of the Health Aspects of Sodium Chloride and Potassium Chloride as Food Ingredients," pp. 1-50 (1979) discusses how much of each of NaCl and KCl is in various commonly consumed foods. Of note, Bajamar Chemical markets (for prescription only sales by pharmacists) powder packets containing 1500 mg of KCI for patients who have a KCI depletion problem, for instance, from taking diuretics.

(All patents mentioned are incorporated by reference.)

Nevertheless, a need still exists for devising other means of treatment for those persons infected with HIV.

### Summary of Invention

Accordingly, the present invention provides a method for the treatment of a human infected with HIV, by administering to the upper gastro-intestinal tract of the human a selected amount of a formulation of sodium chloride. The method comprises (a) administering the sodium chloride formulation to the human's upper gastro-intestinal tract so as to introduce the sodium chloride formulation to the metabolism of the human, and (b) periodically repeating (a) so as to administer a therapeutically effective amount of the sodium chloride formulation to the human's metabolism.

In one embodiment, the sodium chloride formulation is solid, containing sodium chloride, preferably obtained from vacuum granulated sodium chloride that has been compressed into a tablet or that remains as a powder, as further described below. When the sodium chloride formulation is a solid, the solid formulation may contain a majority of sodium chloride, i.e., about 80% to about 100%, more preferably about 95% to about 100% by weight sodium chloride. Nevertheless, amounts less than 80% by weight sodium chloride may desirably be present when potassium ion is also present as discussed below. Furthermore, the solid sodium chloride formulation should be free of a carrier, and/or free of other known medicaments, i.e., AZT, 3TC, and the like, for suppression of the AIDS virus.

In another embodiment, the sodium chloride is in a solution, which formulation is preferably at least 2% by weight sodium chloride. Sodium chloride may be present up to the saturation point in water, i.e., about 32.6% by weight sodium chloride at room temperature (72°F, 22.2°C). Also preferably, the solution formulation is an aqueous solution and should be free of solvents other than water and also free of other known medicaments, i.e., AZT, 3TC, and the like, for suppression of the AIDS virus. Thus, although the solution formulation may consist essentially of water with sodium chloride dissolved in the water, potassium ion also may desirably be present in the solution formulation as discussed below.

In still another embodiment, the sodium chloride formulation includes another ingredient that is the kind of ingredient naturally present in human extracellular fluid and/or human intracellular fluid.

In still another embodiment, the sodium chloride formulation includes another ingredient such as S, P, Zn, Mn, Fe, Cu, Cr, I, Mg, Co, and/or Se.

Accordingly, it is an object of the present invention to provide a treatment for humans infected with HIV.

It is a further object of the present invention that sodium chloride used in the treatment may be orally ingested, and thereby the present invention obviates the drawbacks of intravenous administration.

Some of the objects of the invention having been stated above, others will become evident as the description proceeds, when taken in conjunction with Laboratory Examples as best described below.

### Detailed Description

More specifically, the inventive method involves administration of a formulation of sodium chloride, the chemical formula of which is NaCI, to treat a human who is infected with HIV, including full-blown AIDS. The administration is to the human's upper gastro-intestinal tract in order to introduce NaCI to the human's metabolism.

In one embodiment, the administration is of a solid formulation of NaCl, such as a tablet or powder, that may be orally administered by being swallowed. In addition to oral administration, contemplated also is administration of a solid formulation of NaCI that is intraoral via the mucous membrane of the mouth (such as sublingual or buccal) or transdermal (such as with a skin patch). A good discussion of intraoral administration can be seen in U.S. Patent No. 4,229,447 issued October 21, 1980 to Porter and in U.S. Patent No. 5,504,086 issued April 2, 1996 to Ellinwood and Gupta. A good discussion of transdermal administration can be seen in U.S. Patent No. 5,016,652 issued May 21, 1991 to Rose and Jarvik.

When the formulation of NaCl is in the form of a solid, the solid formulation may contain between about 55% and about 100% by weight, preferably, about 65% and about 100% by weight, and more preferably, about 75% to about 100% by weight, and even more preferably, about 95% to about 100% by weight, NaCI with only trace amounts, if any, present of other ingredients, for instance, other mineral salts such as magnesium chloride or calcium chloride.

However, other ingredients may be present in the solid formulation of NaCl.

For instance, potassium salts and/or other potassium complexes, may be present. Also, other sodium salts and/or sodium complexes may be present. These particular ingredients are the kinds of ingredients naturally present in human extracellular fluid (plasma fluid or interstitial fluid) and/or in human intracellular fluid. As discussed in Review of Medical Physiology, Ganong, 18^{th} ed., Chapter 1 (The General and Cellular Basis of Medical Physiology), pp. 27-28 (1997), human body fluid naturally contains, in addition to sodium chloride, ingredients such as potassium chloride, potassium carbonate, potassium protein complexes, potassium phosphate, sodium carbonate, sodium protein complexes, and sodium phosphate.

In particular, potassium ion may be present in a Na:K ratio by weight of up to about 1:1. For instance, a solid tablet may contain about 57.5% by weight NaCl and 42.5% by weight KCI, or a solid tablet may contain about 46% by weight NaCI, about 34% by weight KCI, and about 20% by weight various other ingredients such as S, P, Zn, Mn, Fe, Cu, Cr, I, Mg, Co, and/or Se. It is noted that these percents of NaCI and KCI result in a Na:K ratio of about 1:1.

The amount of such other ingredients should be less than about 45%, more preferably less than about 35%, and even more preferably less than about 25% by weight, based on the weight of the sodium chloride. Thus, the amount of sodium chloride would be more than about 55%, more preferably more than about 65%, and even more preferably more than about 75%.

Substantially pure solid NaCI naturally occurs as rock salt, also known as the mineral halite. Halite is translucent when pure, but may be white, yellow, red or blue when trace amounts of other minerals are present.

Also, Eli Lilly of Indianapolis, Indiana markets sodium chloride tablets (1000 mg) for salt replacement by oral administration to humans. Moreover, a NaCI tablet that is about 250 mg (which is a size convenient for swallowing) may be employed for the embodiment of the present invention when the NaCI is a solid formulation.

Furthermore, Morton Salt of Chicago, Illinois markets NaCI. Additionally, provided by Morton Salt to its customers is its NaCI product sold under the trademark PUREX®. PUREX® is a granulated NaCI having a mean average crystal size of 430 microns or 360 microns, depending on whether the product is manufactured at Morton Salt's facility in Rittman, Ohio or Silver Springs, New York, respectively. PUREX® should also be useful in the method of the present invention for when administration of a solid formulation of NaCI comprises administration of powder.

For use in the present invention, the solid formulation of NaCI also may be free of any carriers. For instance, typical pharmaceutically acceptable carriers (such as ethanol, glycerol, stearyl alcohol, polyethylene glycol, propylene glycol, and glycerylmonostearate, often used to place a medicament in solution form or emulsion form for administration) need not be used for the present invention, and preferably, are not used, except as described below vis-a-vis solutions of NaCI such as for administration with a feeding tube.

In an alternative embodiment, the NaCI preferably is in a solution formulation, more preferably an aqueous solution, for administration of NaCI to a human's upper gastro-intestinal tract in order to introduce the NaCI to the human's metabolism. For this solution formulation embodiment, in addition to oral administration such as by a human swallowing a solution formulation of NaCI by way of the mouth, contemplated also is administration of a solution formulation to the esophagus, stomach, and/or duodenum, such as by gavage, i.e., by way of a feeding tube. Gavage type of administration is useful for when the HIV has progressed in the person to full blown AIDS, and the person can no longer swallow food, medicine, et cetera, by mouth. Nevertheless, to improve palatability when the solution formulation is swallowed, the solution formulation may contain a flavoring. Suitable flavorings may be selected from the group consisting of sugar, coffee, beer, wine, whiskey, fruit juice, milk, soda, mint, and combinations thereof.

Preferably, only water is employed as the solvent in order to place the NaCI in a solution formulation. Thus, although typical pharmaceutical solvents (such as ethanol, glycerol, stearyl alcohol, polyethylene glycol, propylene glycol, and/or glycerylmonostearate, often used to place a medicament in solution form or emulsion form) may be employed in the embodiment of the present invention involving a solution formulation of NaCl, such pharmaceutical solvents are not desired. Hence, the solution formulation should consist essentially of water with NaCl dissolved in the water.

The solution formulation should be at least 2%, more preferably at least 5%, most preferably at least 10%, by weight NaCI, and may be saturated with NaCI. At room temperature, saturation of NaCI in water is about 32.6% by weight NaCI, and at 0°C is about 35.7% by weight NaCI.

As discussed above vis-a-vis the solid formulation of sodium chloride, other ingredients, such as the kinds of ingredients naturally present in human extracellular fluid (plasma fluid or intestinal fluid) and/or in human intracellular fluid, may be present in addition to sodium chloride in the solution formulation. These are other ingredients such as potassium chloride, potassium carbonate, potassium protein complexes, potassium phosphate, sodium carbonate, sodium protein complexes, and sodium phosphate.

In particular, potassium ion may be, as noted above, present in a Na:K ratio by weight of up to about 1:1. For instance, the non-water components of an aqueous solution formulation of NaCI, based on the weight of the NaCI, may be about 57.5% NaCI and about 42.5% KCI, or may be about 46% NaCI, about 34% KCl, and about 20% various other ingredients such as S, P, Zn, Mn, Fe, Cu, Cr, I, Mg, Co, and/or Se. It is noted that these percents of NaCI and KCI result in a Na:K ratio of about 1:1. Each of the various other ingredients, such as S, P, Zn, Mn, Fe, Cu, Cr, I, Mg, Co, and/or Se, may be individually present in a trace amount up to about 1.8% - 1.9%.

The amount of all such other non-water ingredients should be less than about 45%, more preferably less than about 35%, and even more preferably less than about 25% by weight, based on the weight of the sodium chloride in the solution formulation. Thus, the amount of sodium chloride would be more than about 55%, more preferably more than about 65%, and even more preferably more than about 75%.

Additionally, the solid formulation of NaCI or the solution formulation of NaCI also may be free of any other known medicaments, i.e., AZT, 3TC, and the like, for suppression of the AIDS virus.

For both the solid formulation of NaCI and the solution of NaCI, care should be taken to monitor the amount of potassium present in the patient's blood as potassium is a factor in the clotting of blood and too much can cause the patient to have a problem with hyperkalemia. Nevertheless, when KCI is present, the amount may exceed the 12 to 20 mg average daily intake (see, pp. 38-39 of Fisher, supra), particularly when the patient has a KCI depletion problem (see, Bajamar Chemical's KCI powder packets, supra).

Also, for both the solid formulation of NaCI and the solution formulation of NaCI, the presence of Se, in an amount preferably of at least 120 mcg, is desirable, The reason is that, as is well known, a person with HIV infection often will develop a Se deficiency which is associated with dilated cardiomyopathy. However, the amount of Se should be low enough so that the total daily dosage of Se does not exceed 200 mcg since too much Se is known to cause side effects such as garlic breath, hair loss, and/or nausea.

Administration of the NaCI formulation should be sufficient to provide more than the minimum daily requirement of NaCI according to the National Academy of Sciences, which is a minimum recommendation for Americans of 500 mg/day of sodium (1250 mg/day of NaCI). More preferably, administration of the NaCI formulation should be sufficient to provide more than what the average American chooses to consume (which is 4960 to 6230 mg/day of NaCI according to the U.S. Food and Drug Administration) and should be sufficient to provide more than what the average human of the world's population chooses to consume where salt is readily available (which is 6000 to 11000 mg/day of NaCI as reported by Bertram in "Sodium Halides, Sodium Chloride", Vol. 22, Kirk-Othmer Encyclopedia of Chemical Technology, 4^{th} Ed., p. 370, 1997).

In view of the amounts described in the paragraph above, the person to whom the NaCI formulation is going to be administered should be monitored for a month or so to determine this person's average daily intake of NaCI, and then, the amount administered should be sufficient to be more. More particularly, the amount administered should be at least 250 mg/day more, more preferably 750 mg/day more, and even more preferably 1250 mg/day more NaCI than the particular person's average daily intake. Hence, administration of the NaCI formulation may be done at least 1 time per day, but may be oftener depending on the severity of the HIV infection in the human and the particular average daily intake of NaCI for that human. Hence, administration may be as often as 5 or 6 times per day, or even more. Typically, for most humans, administration once or twice per day is sufficient. However, regardless of the particular human's average daily intake, administration should be sufficiently more than that average daily intake so that the total daily intake is at least 7500 mg/day, more preferably 9500 mg/day, and even more preferably 11500 mg/day NaCI.

Administration of the NaCI formulation should be repeated (daily, twice daily, etc.) On a regular basis for months or even years, and the HIV infection will have been alleviated and possibly eliminated. In other words, the blood will consistently test negative for the presence of HIV infection. Blood tests, such as ELISA or Western Blot, for the presence of HIV infection in a human patient are well known.

For severe cases, administration of the NaCI formulation should continue for the rest of the human's life. Otherwise, HIV infection may take hold again. Even for other humans, after elimination of the HIV infection, administration may be once per day to maintain the human free of the HIV infection.

Although administration of the NaCI formulation to humans suffering from HIV infection, including humans suffering from AIDS illness and/or humans suffering from related conditions such as AIDS related complex (ARC), under conditions which effectively interrupt or suppress activity of the HIV virus, can be accomplished by one or more of several means of administration as described above, in the preferred embodiment, whatever administrative method is chosen should result in circulating levels of the NaCI within a range of about 0.05 uM to about 1.0 uM. A dosage range of about 0.01mg of NaCl/kg of body weight given every 4 hours is considered a virustatic range in most large mammals. In order to achieve this, the preliminary dosage range for oral administration, is slightly broader, being for example, 0.005-0.25mg of NaCI/kg of body weight given every 4 hours. It is recognized that modifications might need to be made in individual patients to ameliorate or to forestall toxic side effects.

Regardless, the dosage of NaCl must be less than the toxicity measured by a standard called LD₅₀, namely the dosage that is lethal for 50% of the population. Furthermore, the dosage must also be less than the toxicity measured by a standard called TCLo, namely the dosage for oral consumption that is the lowest dosage that has produced toxic effects in humans. As is well known, the TDLo for NaCI in humans is 12357 mg of NaCI/kg of body weight/day for 23 days of continuous oral consumption.

Laboratory Examples

The following is representative of what would be expected to occur when persons would be treated in accordance with the present invention. All subjects would be first monitored to determine the average daily intake of NaCI for each. Administration to each respective subject always is sufficient to provide at least 250mg of NaCl per day more than the respective subject's average daily intake.

Example I (Testing with Aqueous NaCI)

Test Subject No. 1. A representative person would be an adult male Caucasian who tests positive in a blood test for HIV infection. Once per day, he would drink 8 ounces of an aqueous solution of 2% by weight NaCI in tap water (about 560 mg of NaCI). This would continue for 5 months, and he should then test negative in a blood test for HIV infection. He stops the treatment, and within 1 month he may test positive in a blood test for HIV infection. Thus, treatment would resume, and in a few months, he should test negative again.

Test Subject No. 2. The method is repeated in the same manner of administration of 2% by weight NaCl in tap water (about 560 mg of NaCI), as with the above-noted male test subject no. 1, but instead with a representative teenaged female person who tests positive in a blood test for HIV infection. Treatment at least once per day would be continued indefinitely and the results should be that she tests negative in a blood test for HIV infection by about month 5 of the treatment.

Example II

(Testing with solid NaCI)

12 representative persons would be treated by the inventive method, but with a 250 mg tablet of solid NaCI (except certain other ingredients also are incorporated into the tablets, for a total of 250 mg, for test subject nos. 11 and 12, as further discussed below).

No complications result from the use of the NaCI tablets by any of the 12 subjects.

In general, the testing of each of the 12 subjects would be conducted as follows:

A certain number of times per day as indicated, the subject would swallow a NaCI tablet with a 5 ounce glass of tap water.

The administration would be planned to continue for the remainder of the subjects' lives. Depending on the mildness to severity of the HIV condition, generally at some point between 2 months and 5 months, the subjects should intermittently begin to test negative in a blood test administered monthly for HIV infection, and usually within another 2 to 3 months, each subject should consistently test negative. Most subjects would continue with the administration of NaCI tablets to maintain the negative blood test.

12 representative subjects would be as follows:

Test Subjects Nos. 1, 2, and 3. These representative persons would be young adult females each of whom is afflicted with HIV infection. In the past, each subject would have tried various oral antibiotics for treatment of HIV infection with poor results.

At the time of testing with the NaCI tablet, each subject does not take any medication orally and does not use any medication topically for treatment of HIV infection. Each subject would start once daily administration with the NaCI tablet. After that continues for 6 months, each subject should consistently test negative in a blood test administered monthly for HIV infection. The 3 subjects would continue administration once per day with a tablet of NaCI.

Test Subjects Nos. 4 and 5. Each of these representative persons would be a young adult male with HIV infection that had progressed to the stage of full blown AIDS. In the past, each subject has tried various oral antibiotics, as well as topical antibiotics, with poor results.

At the time of testing with the NaCl tablet, each subject does not take any medication orally and does not use any medication topically. Each subject would start twice daily administration with the NaCI tablets. After that continues for 6 months, each subject may still consistently test positive in a blood test administered monthly for HIV infection due to the full blown AIDS. Thus, administration would be increased to 8 tablets per day for each subject. After 6 more months, each subject should consistently test negative in a blood test administered monthly for HIV infection. The 2 subjects would continue 8 tablets per day administration of the NaCI tablets.

Test Subjects Nos. 6 and 7. These representative persons would be middle-aged adult males with HIV infection. Both would report that when work is stressful, they notice fatigue for long periods of time. In the past, both would have tried various oral antibiotics for treatment of HIV infection. After 7 months of use once daily of the NaCI tablet, each should consistently test negative in a blood test administered monthly for HIV infection.

Test Subject No. 8. This representative person would be a male senior citizen with HIV infection. After 8 months of using a NaCI tablet once daily, he should consistently test negative in a blood test administered monthly for HIV infection.

Test Subject No. 9. This representative person would be a female senior citizen with HIV infection. After 9 months of swallowing a NaCI tablet once per day, she would still test positive in a blood test administered monthly for HIV infection and show little improvement as compared to other medications previously used for treatment of her HIV infection. However, she would not achieve better results because she would also indicate that she forgets to be consistent in daily use of NaCI.

Test Subject No. 10. This representative person would be a female senior citizen with HIV infection that had progressed to the stage of full blown AIDS. After 10 months of 10 times daily use of the NaCI tablets she should consistently test negative in a blood test administered monthly for HIV infection. She would continue with the 10 tablets of NaCI per day.

Test Subjects Nos. 11 and 12. Testing as per subject no. 10 would be repeated with two females of comparable age and stage of infection.

This time for the first female, the tablets would also contain KCI sufficient to provide a weight ratio in each 250 mg tablet of Na:K of about 1:1, namely about 57.5% NaCI and about 42.5% KCI.

For the second female, the tablets would again contain KCl, but also would contain S, P, Zn, Mn, Fe, Cu, Cr, I, Mg, Co, and Se, namely about 46% NaCI, about 34% KCI, and about 20% of the others, with at least 120 mcg Se per 250 mg tablet. Similar results should be obtained.

It will be understood that various details of the invention may be changed without further departing from the scope of the invention. Furthermore, the foregoing description is for the purpose of illustration only, and not for the purpose of limitation -- the invention being defined by the claims.

What is Claimed is:

1. (Cancelled.) A method for providing sodium chloride to a human having HIV infection by administering to the upper gastro-intestinal tract of the human a selected amount of a formulation of sodium chloride, said method comprising:
(a) administring the sodium chloride formulation to the human's upper gastro-intestinal tract so as to introduce the sodium chloride formulation to the metabolism of the human; and
(b) periodically repeating (a), so as to administer a therapeutically effective amount of the sodium chloride formulation to the human's metabolism.

2. (Cancelled.) The method of claim 1, wherein the sodium chloride formulation is free of having other medicaments incorporated therewith for treatment of HIV infection.

3. (Cancelled.) .The method of claim 1, wherein steps (a) and (b) are accomplished at least once per day.

4. (Cancelled.) The method of claim 1, wherein the amount of the sodium chloride formulation administered is sufficient to provide at least about 250 mg per day more sodium chloride than the human's average daily intake for sodium chloride, as determined after monitoring the human for about 1 month.

5. (Cancelled.) The method of claim 4, wherein the amount of the sodium chloride formulation administered and the average daily intake for sodium chloride provide at least 7500 mg/day of sodium chloride.

6. (Cancelled.) The method of claim 1, wherein the sodium chloride formulation is a mixture with a form of potassium in a weight ratio amount of Na:K up to about 1:1.

7. (Cancelled.) The method of claim 6, wherein the mixture contains up to about 20% by weight of another ingredient selected from the group consisting of S, P, Zn, Mn, Fe, Cu, Cr, I, Mg, Co, Se, and combinations thereof.

8. (Cancelled.) The method of claim 1, wherein the sodium chloride formulation is free of having other mineral salts incorporated therewith except for trace amounts thereof.

9. (Cancelled.) The method of claim 1, wherein the sodium chloride formulation is in a form selected from the group consisting of a solid formulation of sodium chloride and a solution formulation of sodium chloride.

10. (Cancelled.) The method of claim 9, wherein the solid formulation contains from about 55% to about 100% of sodium chloride.

11. (Cancelled.) The method of claim 10, wherein the solid formulation contains from about 75% to about 100% by weight sodium chloride.

12. (Cancelled.) The method of claim 9, wherein the solid formulation of sodium chloride is free of having a carrier incorporated therewith.

13. (Cancelled.) The method of claim 9, wherein the solid formulation of sodium chloride is selected from the group consisting of a tablet, a powder, and a combination thereof.

14. (Cancelled.) The method of claim 9, wherein administration of the solid formulation of sodium chloride is administration selected from the group consisting of oral, sublingual, buccal, transdermal, and a combination thereof.

15. (Cancelled.) The method of claim 9, wherein the solution formulation of sodium chloride contains at least about 2% by weight sodium chloride.

16. (Cancelled.) The method according to claim 9, wherein the solution formulation of sodium chloride is aqueous.

17. (Cancelled.) The method according to claim 9, further including a flavoring in the solution formulation of sodium chloride to improve palatability.

18. (Cancelled.) The method according to claim 17, wherein the flavoring is selected from the group consisting of sugar, coffee, beer, wine, whiskey, fruit juice, milk, soda, mint, and combinations thereof.

19. (Cancelled.) The method of claim 9, wherein administration of the solution formulation of sodium chloride is administration selected from the group consisting of oral, gavage, and a combination thereof.

20. (Cancelled.) The method according to claim 1, wherein the administration to the upper gastro-intestinal tract is by way of a portion of the upper gastro-intestinal tract selected from the group consisting of a mouth, an esophagus, a stomach, a duodenum, and a combination thereof.

21. (Cancelled.) The method according to claim 1, further including a minor amount of another ingredient selected from the group consisting of potassium chloride, potassium carbonate, potassium protein complexes, potassium phosphate, sodium carbonate, sodium protein complexes, sodium phosphate, and combinations thereof, wherein the total minor amount of said other ingredients is less than about 45% by weight, based on the weight of the sodium chloride.

22. (New) A method for providing sodium chloride to a human having HIV infection by administering to the upper gastro-intestinal tract of the human a selected amount of a formulation of sodium chloride, said method comprising:
(a)administering the sodium chloride formulation to the human's upper gastro-intestinal tract so as to introduce the sodium chloride formulation to the metabolism of the human, wherein the amount of the sodium chloride in the sodium chloride formulation administered is (i) sufficient to provide more sodium chloride than the human's average daily intake for sodium chloride, as determined after monitoring the human for about 1 month, (ii) less than a toxic amount measured by TCLo, the dosage for oral consumption that is the lowest dosage of sodium chloride that has produced toxic side effects in humans, and (iii) less than a toxic amount measured by LD50, the dosage of sodium chloride that is lethal for 50% of the human population;
(b)periodically repeating (a), so as to administer a therapeutically effective amount of the sodium chloride formulation to the human's metabolism; and
(c)achieving alleviation of the HIV infection.

23. (New) The method of claim 22, wherein the sodium chloride formulation is free of having other medicaments incorporated therewith for treatment of HIV infection.

24. (New) The method of claim 22, wherein steps (a) and (b) are accomplished at least once per day.

25. (New) The method of claim 22, wherein the amount of the sodium chloride formulation administered is sufficient to provide at least about 250 mg per day more sodium chloride than the human's average daily intake for sodium chloride, as determined after monitoring the human for about 1 month.

26. (New) The method of claim 25, wherein the amount of the sodium chloride formulation administered and the average daily intake for sodium chloride provide at least 7500 mg/day of sodium chloride.

27. (New) The method of claim 22, wherein the sodium chloride formulation is a mixture with a form of potassium in a weight ratio amount of Na:K up to about 1:1.

28. (New) The method of claim 27, wherein the mixture contains up to about 20% by weight of another ingredient selected from the group consisting of S, P, Zn, Mn, Fe, Cu, Cr, I, Mg, Co, Se, and combinations thereof.

29. (New) The method of claim 22, wherein the sodium chloride formulation is free of having other mineral salts incorporated therewith except for trace amounts thereof.

30. (New) The method of claim 22, wherein the sodium chloride formulation is in a form selected from the group consisting of a solid formulation of sodium chloride and a solution formulation of sodium chloride.

31. (New) The method of claim 30, wherein the solid formulation contains from about 55% to about 100% of sodium chloride.

32. (New) The method of claim 31, wherein the solid formulation contains from about 75% to about 100% by weight sodium chloride.

33. (New) The method of claim 30, wherein the solid formulation of sodium chloride is free of having a carrier incorporated therewith.

34. (New) The method of claim 30, wherein the solid formulation of sodium chloride is selected from the group consisting of a tablet, a powder, and a combination thereof.

35. (New) The method of claim 30, wherein administration of the solid formulation of sodium chloride is administration selected from the group consisting of oral, sublingual, buccal, transdermal, and a combination thereof.

36. (New) The method of claim 30, wherein the solution formulation of sodium chloride contains at least about 2% by weight sodium chloride.

37. (New) The method according to claim 30, wherein the solution formulation of sodium chloride is aqueous.

38. (New) The method according to claim 30, further including a flavoring in the solution formulation of sodium chloride to improve palatability.

39. (New) The method according to claim 38, wherein the flavoring is selected from the group consisting of sugar, coffee, beer, wine, whiskey, fruit juice, milk, soda, mint, and combinations thereof.

40. (New) The method of claim 30, wherein administration of the solution formulation of sodium chloride is administration selected from the group consisting of oral, gavage, and a combination thereof.

41. (New) The method according to claim 22, wherein the administration to the upper gastro-intestinal tract is by way of a portion of the upper gastro-intestinal tract selected from the group consisting of a mouth, an esophagus, a stomach, a duodenum, and a combination thereof.

42. (New) The method according to claim 22, further including a minor amount of another ingredient selected from the group consisting of potassium chloride, potassium carbonate, potassium protein complexes, potassium phosphate, sodium carbonate, sodium protein complexes, sodium phosphate, and combinations thereof, wherein the total minor amount of said other ingredients is less than about 45% by weight, based on the weight of the sodium chloride.
